(19) United States
(12) Patent Application Publication
(10) Pub. No.: US 2009/0308764 A1
(43) Pub. Date: Dec. 17, 2009

(54) Hemostatic bandage kit and method of use

(76) Inventors: Kevin R. Lee, Walled Lake, MI (US); Allison Lee, Walled Lake, MI (US)

Correspondence Address: T.L. GARRETT, PLC, P.O. BOX 1065, FENTON, MI, 48430, US

(21) Appl. No.: 12/310,942

(22) PCT Filed: Sep. 13, 2007
(86) PCT No.: PCT/US2007/020078

§ 371 Date: Mar. 12, 2009

Related U.S. Application Data
(63) Continuation of application No. 11/895,977, filed on Aug. 28, 2007.
(60) Provisional application No. 60/844,441, filed on Sep. 14, 2006.
(60) Provisional application No. 60/844,441, filed on Sep. 14, 2006.

(51) Int. Cl. A61B 19/02; B65D 25/08; A61L 15/00

(52) U.S. Cl. 206/219; 206/440; 602/48

(57) ABSTRACT

The hemostatic bandage kit and associated method of use operates to yield a rapidly deployable hemostatic wound treatment to enable medical personnel to more quickly respond to traumatic injuries involving massive blood loss, thereby saving lives. The hemostatic bandage kit does not require a medical facility, or alternatively, medical personnel or others in the field, to separately store or carry the components necessary to create a hemostatic wound treatment. All of the components are cooperatively stored in an integrated kit that is easily transported, stored, and deployed. The components include an inner membrane retaining a solvent therein, and an outer membrane retaining an absorptive material and a clotting agent, wherein the inner membrane is adapted to rupture and mix the solvent with the clotting agent and to saturate the absorptive material for application to a wound.

### REFERENCE TO RELATED APPLICATION

The present PCT application claims priority benefit of U.S. Non-Provisional patent application Ser. No. 11/895,977, filed Aug. 28, 2007, titled “HEMOSTATIC BANDAGE KIT AND METHOD OF USE”, the disclosure of which is incorporated herein by reference in its entirety, which claims priority benefit of U.S. Provisional Patent Application No. 60/844,441, filed Sep. 14, 2006, titled “HEMOSTATIC BANDAGE KIT AND METHOD OF USE”, the disclosure of which is incorporated herein by reference in its entirety.

### FIELD OF THE INVENTION

The present invention generally relates to a hemostatic bandage kit and more particularly, relates to a kit that includes an absorptive material and a clotting agent having more than one component which are stored separately within the kit, and not mixed together until the bandage is ready to be used.

### BACKGROUND

Traumatic bodily injury leads to many complications. One of the greatest threats to those who have sustained a major injury is the loss of blood. In many cases, the body's ability to stem the flow of blood to the site of an injury is not sufficient to allow the injured to retain an adequate supply of blood. Without an adequate supply of blood, the body is unable to function, resulting in morbidity and mortality to the wounded.

Bandages and absorbent materials are both means of stifling the flow of blood from the site of an injury. Often, the use of a bandage at the site of a wound will allow a sufficient amount of blood to gather, allowing the body's natural blood-clotting processes to take effect, thereby stopping the flow of blood. However, if the wound is sufficiently large, the body's internal mechanisms are unable to cope with the outflow of blood, even when an absorbent material or bandage is placed at the site of the wound.

Clotting agents have been developed which aid the body in its clotting efforts at the site of an injury. Many of these clotting agents are derived from human or animal products. Due to the nature of these products, they must be processed in such a way as to prevent their decay or degradation over time. These conditions are prevented by lyophilizing, or freeze-drying, the clotting agent. However, in order for the clotting agent to be effective in treating an injury, it must be combined with a solvent.

Existing devices using a clotting agent and an absorptive material in the form of a kit to effect hemostasis have time delays associated with saturation of the absorptive material with the mixing of the clotting agent and solvent. Additionally, existing devices may allow for only one side of the material to be applied to the wound.

Therefore, what is needed is a hemostatic bandage kit that will quickly and thoroughly saturate an absorptive material, allowing it to be quickly accessed and effectively used on all types of injuries requiring rapid hemostasis.

### SUMMARY

A hemostatic bandage kit that contains components necessary to supply an absorptive material that is thoroughly saturated with a clotting agent and a solvent to rapidly create hemostasis at the site of an injury. The hemostatic bandage kit of the present invention provides a sterile, self-contained device for both the storage and dispersion of the clotting agent and solvent, providing simplicity of use, reduced storage requirements, and extremely rapid deployment.

In an embodiment of the invention the hemostatic bandage kit includes an inner membrane that operates to sealingly retain a solvent therein; a solvent retained within the inner membrane; an absorptive material; a clotting agent adapted to dissolve within the solvent and an outer membrane having an interior region that operates to sealingly retain the absorptive material and the clotting agent therein. The clotting agent dissolves within the solvent to form a clotting solution when the inner membrane ruptures and communicates the solvent to the outer membrane interior region thereby saturating the absorptive material with the clotting solution. Each component of the kit is sterilized prior to use, including the interior region of the outer membrane and the inner membrane.

A method of using a hemostatic bandage kit includes squeezing the outer membrane with sufficient force to rupture the inner membrane; shaking the kit once the inner membrane is ruptured, allowing the solvent and clotting agent to mix and propagate throughout the outer membrane, thereby saturating the absorptive material; and accessing an interior region of the outer membrane to retrieve the saturated absorptive material.

In one embodiment of the invention a perforated portion of the outer membrane is opened so as to allow a user of the kit to remove the bandage from the outer membrane interior region.

### BRIEF DESCRIPTION OF THE DRAWINGS

These and other objects, features and advantages of the present invention will become apparent from the following detailed description and the appended drawings in which:

FIG. 1 is a perspective view of a hemostatic bandage kit in accordance with one embodiment of the invention.

FIG. 2 is a cross-sectional view of the hemostatic bandage kit shown in FIG. 1 taken across Line 2-2.

FIG. 3 is a cut-away perspective view of the hemostatic bandage kit including a partition forming two compartments within an outer membrane to house elements of the kit in accordance with one embodiment of the invention.

FIG. 4 is a cross-sectional view of the hemostatic bandage kit shown in FIG. 3 taken across line 4-4.

FIG. 5 is a cross-sectional view of the hemostatic bandage kit similar to the kit shown in FIG. 3 without an inner membrane disposed in a second compartment.

FIG. 6 is a perspective in use view of an opened hemostatic bandage kit exposing an absorptive material and an outer membrane interior region in accordance with one embodiment of the invention.

### DETAILED DESCRIPTION

The present invention is directed to a hemostatic bandage kit.

The hemostatic bandage kit and associated method of use operates to yield a rapidly deployable hemostatic wound treatment to enable medical personnel to more quickly respond to traumatic injuries involving massive blood loss, thereby saving lives.

The hemostatic bandage kit does not require a medical facility, or alternatively, medical personnel or others in the field, to separately store or carry the components necessary to create a hemostatic wound treatment. All of the components are cooperatively stored in an integrated kit that is easily transported, stored, and deployed. A user of the hemostatic bandage kit is able to easily and effectively prepare a wound treatment with a minimum of time or training, which may be beneficial in situations where persons without formal medical training may be required to treat wounds, such as soldiers on a battlefield. As defined herein, a user of the kit may be an end-user, such as a patient, or may be any person accessing the contents of the kit, such as medical personnel.

Thus, the hemostatic bandage kit allows a wound to be rapidly treated in such a fashion as to establish hemostatis quickly, thereby minimizing blood loss and consequently enhancing the survival rate for injuries that may otherwise be fatal.

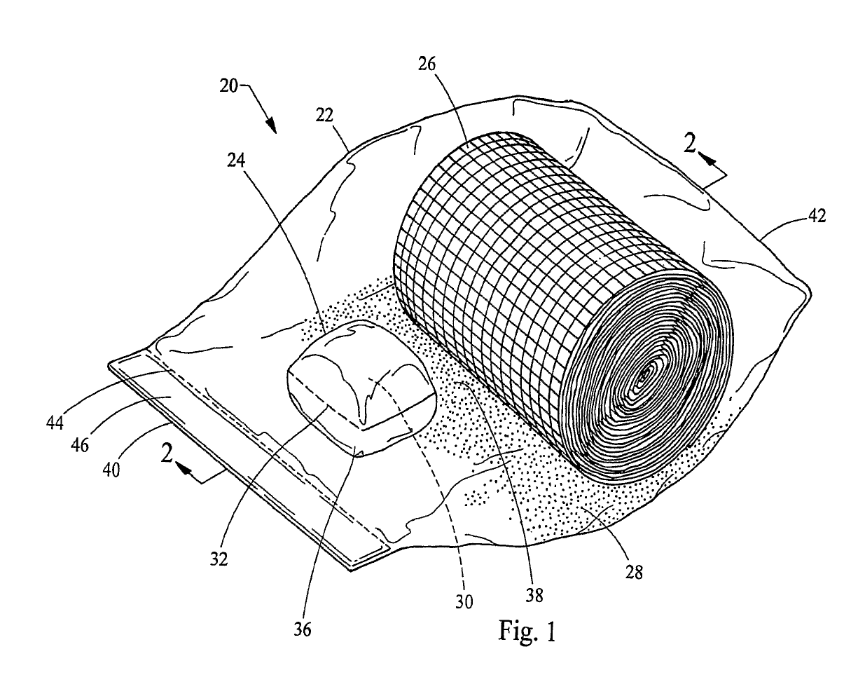

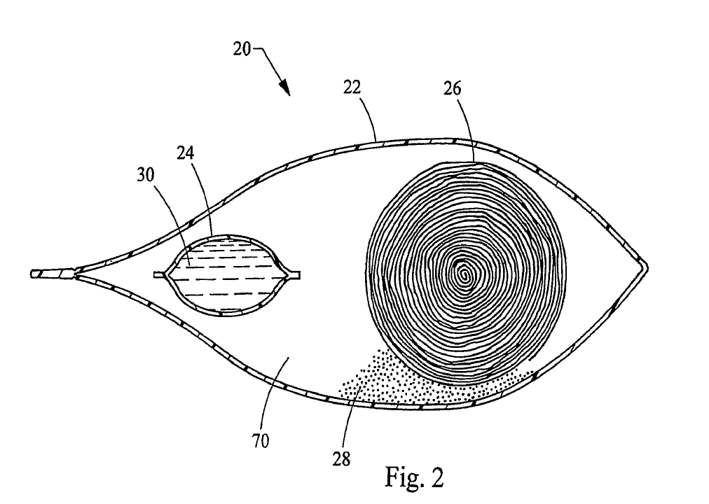

With reference now to the figures, FIG. 1 more particularly, FIG. 1 shows a perspective view of hemostatic bandage kit 20, in accordance with one embodiment of the invention. FIG. 2 illustrates a cross-sectional view of the kit 20 shown in FIG. 1.

As shown in FIGS. 1-2, the kit 20 includes an inner membrane 24 that operates to sealingly retain a solvent therein; a solvent 30 retained within the inner membrane 24; an absorptive material 26; a clotting agent capable of dissolving within the solvent and an outer membrane 22 having an interior region 70 that operates to sealingly retain the absorptive material 26 and the clotting agent therein, wherein the clotting agent 28 dissolves within the solvent 30 to form a clotting solution when the inner membrane 24 ruptures and communicates the solvent to the outer membrane interior region for saturation of the absorptive material with the clotting solution.

As defined herein, the absorptive material 26 may be any material capable of absorbing and retaining liquids and that may be topically applied to the skin of or packed within a wound on a patient. In the non-limiting embodiments shown in FIGS. 1-6, the absorptive material may be any conforming dressing or bandage that has come or may come into existence that is capable of being applied to a large surface area of a patient's body, such as a wound inflicted from a battlefield injury. In a non-limiting embodiment, the absorptive material may be in the form of a flat sheet or roll of gauze.

The clotting agent 28 may be any type of clotting agent such as, but not limited to thrombin, which has come or may come into existence capable of being activated upon mixing with a solvent. The clotting agent may be granulated, in powdered form or another formulation suitable for release into solution. Additionally, the clotting agent may be loosely dispersed throughout the interior region 70 of the outer membrane, or alternatively, the clotting agent may be dispersed throughout the absorptive material to promote homogenous saturation of the material when mixed with the solvent.

The solvent may be any type of solvent or dilutent that mixes with the clotting agent to dissolve and/or activate the hemostatic properties of the clotting agent.

In one non-limiting embodiment wherein the clotting agent 28 is powdered thrombin, the solvent 30 may be in the form of a saline solution.

Additionally, it is contemplated that additional components may be added to the solvent within the inner membrane 24, within a separate retaining device retained within the outer membrane 22, or to the clotting agent 28 within the outer membrane to further enhance the effectiveness of the kit 20. For example, an antiseptic, anesthetic or an antibiotic component or a combination thereof may be added in such a manner to the kits shown in FIGS. 1-6.

Additionally, any components that have come or may come into existence further promoting or providing for effective wound care treatment may be incorporated into any of the kits shown in FIGS. 1-6.

As shown in FIG. 1, the inner membrane 24 is disposed within the outer membrane 22 and forms a sealed container for retaining the solvent 30. The inner membrane 24 has a weak region 32, wherein the weak region is adapted to separate or rupture when a predefined external force is applied inwardly thereto, thus allowing the inner membrane 24 to rupture and communicate contents disposed within the inner membrane 24 to an interior region of the outer membrane 22.

In one embodiment of the invention, the weak region 32 may be a scored portion that forms a perforation 32 along a surface of the inner membrane 24. It is contemplated that any suitable conventional method of opening the inner membrane 24 of any of the kits 20, 50, and 72 as is known to a skilled artisan may be used and should not be limited to the perforated structure and method as described herein.

In one embodiment of the invention, the inner membrane may form a bag that may include one or more layers, at least one inner and at least one outer layer, wherein at least one of the inner layer and the outer layer has at least one weakened spot formed therewith and may further operate to define at least one perforated region capable of rupturing when an external force is applied thereto to an interior region of the bag outer layer or to an interior region of the outer membrane.

The weak region 32 may be located anywhere along a surface of the inner membrane 24. In a non-limiting embodiment shown in FIG. 1, the weak region 32 is located along a first edge 36 of the inner membrane 24. Alternatively, the weak region 32 may be located along a second edge 38 of the inner membrane 24.

The outer membrane 22 has an associated scored or perforated portion 44 adapted to be opened to expose the contents of the interior region 70 of the outer membrane 22 when desired. In one embodiment of the invention, a removable seal 46 may be pulled to tear the outer membrane 22 open along the perforated portion 44 and to thereby expose the outer membrane interior region 70. When the outer membrane 22 is opened, a user of the kit may easily access the saturated absorptive material 26 for application of the absorptive material 26 to a desired area of a patient's body, such as to a patient's wound.

The outer and inner membranes 22, 24 may be formed of any resilient and liquid impermeable material that has come or may come into existence and that does not substantially bond with or adhere to a clotting agent or a clotting agent solution, wherein not substantially bonding is defined herein as not interfering with the effectiveness of the clotting agent or clotting agent solution.

A method of using a hemostatic bandage kit provides the steps of squeezing the outer membrane with sufficient force to rupture the inner membrane; shaking the kit once the inner membrane is ruptured, allowing the solvent and clotting agent to mix and propagate throughout the outer membrane, thereby saturating the absorptive material; and accessing an interior region of the outer membrane to retrieve the saturated absorptive material.

When a sufficiently strong external force is applied to the kit 20 depicted in FIG. 1, the inner membrane 24 ruptures, allowing the solvent 30 to flow from the inner membrane 24 into an interior region of the outer membrane 22, wherein the solvent 30 then mixes with the clotting agent 28 and dissolves the clotting agent. The kit 20 is then shaken by the user, thereby forming a homogenous mixture of the solvent 30 and the clotting agent 28 and substantially simultaneously saturating the absorptive material 26.

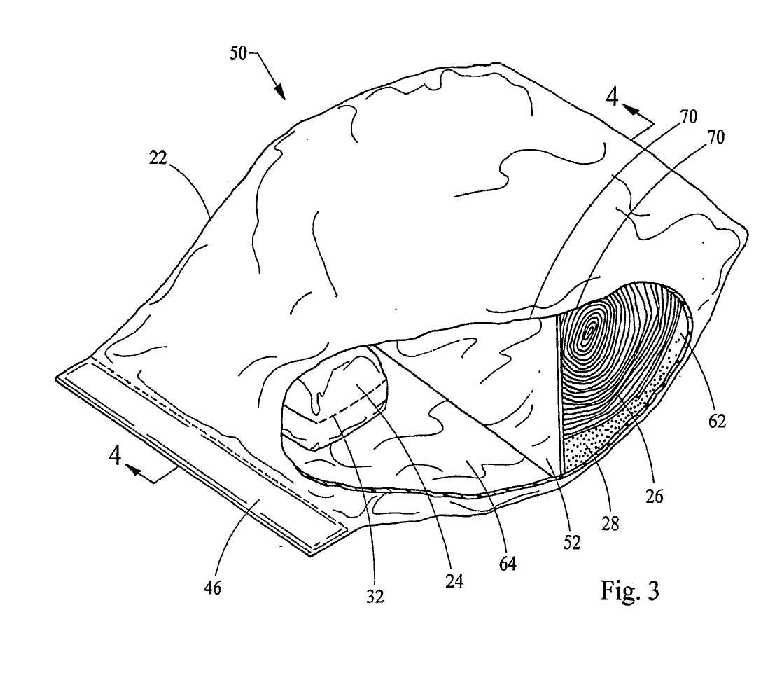

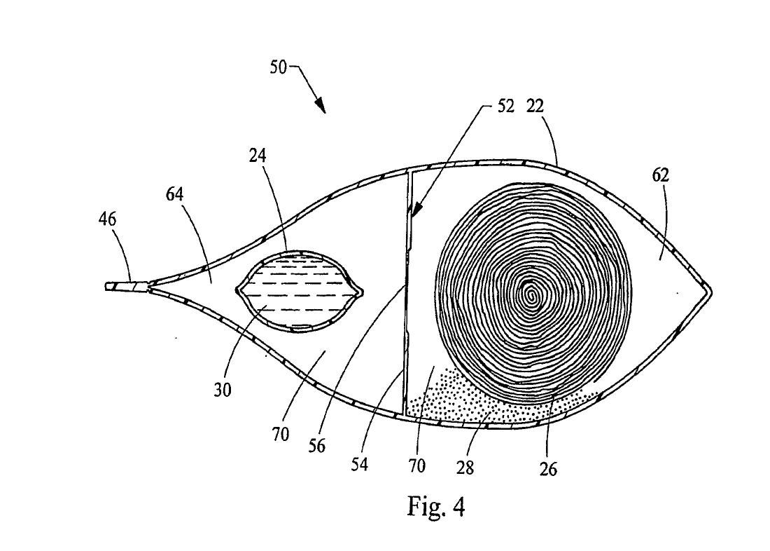

FIG. 3 shows a partial perspective cut-away view of a hemostatic bandage kit 50 in accordance with one embodiment of the invention. FIG. 4 shows a cross-sectional view of the hemostatic bandage kit 50. The kit 50 is similar in structure to the kit 20 but has an additional partition 52 that divides the interior region 70 into a first compartment 62 that retains both the absorptive material 26 and the clotting agent 28 therein and a second compartment 64 that retains the inner membrane 24 therein. The partition 52 is formed by a wall having a varying thickness, wherein the wall has a thicker width portion 52 and a thinner width portion 56, wherein the thinner width portion 56 opens to communicate the contents of the first compartment into the second compartment when a sufficient external force is applied thereto.

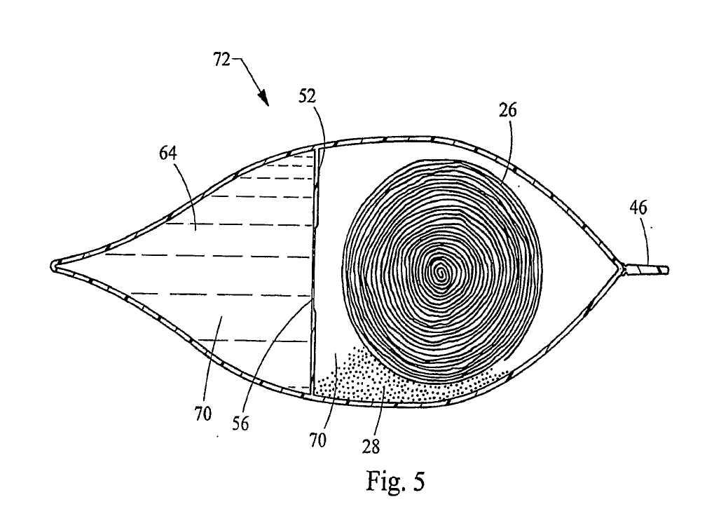

In another embodiment of the invention, a kit 72 is shown in FIG. 5. The kit 72 includes a second compartment 64 that retains the solvent without use of the inner membrane 24 and thus, does not include the inner membrane 24. The absorptive material 26 may be removed from the outer membrane interior region 70 by tearing away the removable seal 46 to expose the region 70.

The embodiments shown in FIGS. 3, 4, and 5 operate in such a manner, such that when the kit 50, and 72 respectively, is exposed to a sufficient external force, the thinner width portion 56 of the internal barrier 52 ruptures, allowing the solvent 30 present in the second compartment 64 to be communicated into the first compartment 62. The user then shakes the kit 50, and 72 respectively, thereby homogeneously mixing the solvent 30 with the clotting agent 28 and saturating the absorptive material 26 with both the solvent 30 and the clotting agent 28.

As shown in FIGS. 1-6, the outer membrane 22 associated with kits, 20, 50, and 72 respectively each have the outer membrane 22 which has a removable seal 46 adapted to be removed by a user of the kits 20, 50, and 72, respectively when desired and a perforated portion 44 formed therewith.

Alternatively, the perforated portion 44 may be formed anywhere along the outer membrane, such as, but not limited to along an outer membrane first or second edge 40, 42, respectively. The perforated portion 44 includes a removable seal 46 adapted to be separated from the first edge 40, thereby allowing the saturated absorptive material 26 to be removed from the outer membrane 22.

It is contemplated that any suitable conventional method of accessing the inner region 70 of any of the kits 20, 50, and 72 as is known to a skilled artisan may be used and should not be limited to the seal and perforated structure and method as described herein.

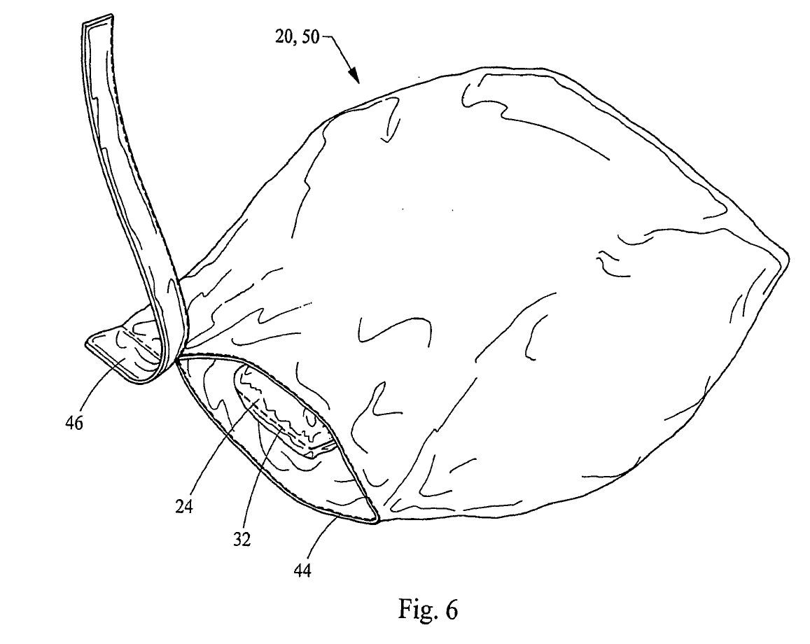

FIG. 6 depicts a method of opening the outer membrane 22 of the embodiments depicted in FIGS. 1-5. The removable seal 46 is shown being torn away from the perforated portion 44 and the absorptive material 26 can be seen protruding from the outer membrane 24.

In another embodiment of the invention, it is contemplated that an additional compartment or bag 80 housing additional kit enhancing components that enhance the overall effectiveness of the kit may be added interiorly or exteriorly to any one of the kits 20, 50, or 72. In a non-limiting embodiment, the bag 80 may include a pair of gloves 82, preferably sterile gloves, peripherally attached to an exterior portion of the outer membrane 22 such that a user of any of the kits 20, 50, 72 may readily open the glove-retaining bag 80, and access the gloves either before or after opening any one of the sterile kits 20, 50, 72, respectively.

The present invention generally relates to a hemostatic bandage kit and method of use. A skilled artisan will appreciate that the foregoing examples are illustrative only, and not limitative of the scope of the technology.

While several aspects have been presented in the foregoing detailed description, it should be understood that a vast number of variations exist and these aspects are merely an example, and it is not intended to limit the scope, applicability or configuration of the invention in any way. Rather, the foregoing detailed description provides those of ordinary skill in the art with a convenient guide for implementing a desired aspect of the invention and various changes can be made in the function and arrangements of the aspects of the technology without departing from the spirit and scope of the appended claims.

What is claimed is:

1. A hemostatic bandage kit comprising:
an inner membrane that operates to sealingly retain a solvent therein;
a solvent retained within the inner membrane;
an absorptive material;
a clotting agent capable of dissolving within the solvent; and
an outer membrane having an interior region that operates to sealingly retain the absorptive material and the clotting agent therein.

2. The hemostatic bandage kit of claim 1, wherein the clotting agent dissolves within the solvent to form a clotting solution when the inner membrane ruptures and communicates the solvent to the outer membrane interior region, thereby homogeneously saturating the absorptive material with the clotting solution.

3. The hemostatic bandage kit of claim 2, wherein the clotting solution does not substantially bond with the outer membrane.

4. The hemostatic bandage kit of claim 1, wherein the clotting agent comprises:
thrombin.

5. The hemostatic bandage kit of claim 1, wherein the solvent comprises:
a saline solution.

6. The hemostatic bandage kit of claim 1, wherein the inner membrane comprises:
at least one weak region, wherein the weak region is adapted to rupture when a predefined external force is applied inwardly thereto, thus allowing the inner membrane to rupture and communicate contents disposed within the inner membrane to an interior region of the outer membrane.

7. The hemostatic bandage kit of claim 6, wherein the weak region comprises:
at least one scored portion that forms a perforation along a surface of the inner membrane.

8. The hemostatic bandage kit of claim 1, wherein the outer membrane comprises:
an associated perforated portion adapted to be opened to expose contents of the interior region of the outer membrane.

9. The hemostatic bandage kit of claim 8, wherein the outer membrane further comprises:
a removable seal that when pulled, operates to tear the outer membrane open along the associated perforated portion and to thereby expose the outer membrane interior region.

10. The hemostatic bandage kit of claim 1, wherein the absorbtive material comprises:
a conforming dressing.

11. The hemostatic bandage kit of claim 1, further comprising:
a resilient and liquid impermeable material forming the inner membrane.

12. The hemostatic bandage kit of claim 1, further comprising:
a resilient and liquid impermeable material forming the outer membrane.

13. The hemostatic bandage kit of claim 1, further comprising:
an antiseptic component.

14. The hemostatic bandage kit of claim 1, further comprising:
an anesthetic component.

15. The hemostatic bandage kit of claim 1, further comprising:
an antibiotic component.

16. A hemostatic bandage kit comprising:
an inner membrane that operates to sealingly retain a solvent therein;
a solvent retained within the inner membrane;
an absorptive material;
a clotting agent capable of dissolving within the solvent; and
an outer membrane having an interior region that operates to sealingly retain the absorptive material and the clotting agent therein, wherein the clotting agent dissolves within the solvent to form a clotting solution when the inner membrane ruptures and communicates the solvent to the outer membrane interior region, thereby substantially simultaneously homogeneously saturating the absorptive material with the clotting solution.

17. The hemostatic gauze kit of claim 16, further comprising:
a glove-retaining bag adapted to sealingly retain a pair of sterile gloves; and
a pair of sterile gloves peripherally attached to an exterior portion of the outer membrane such that a user of the kit may readily open the glove-retaining bag and access the gloves either before or after opening the kit.

18. A method of using a hemostatic bandage kit comprising:
squeezing an outer membrane retaining an absorptive material and a clotting agent with sufficient force to rupture an inner membrane; shaking the kit once the inner membrane is ruptured;
allowing a solvent communicated from the inner membrane to the outer membrane and a clotting agent retained within the outer membrane to mix and propagate throughout the outer membrane to form a clotting solution; saturating the absorptive material with the clotting solution; and
accessing an interior region of the outer membrane to retrieve the saturated absorptive material.

19. The method of using the hemostatic bandage kit of claim 18, further comprising:
forming a homogeneous mixture of the solvent and the clotting agent when the solvent is communicated from the inner membrane to the outer membrane.

20. The method of using the hemostatic bandage kit of claim 19, further comprising:
saturating the absorptive material substantially simultaneously when the homogeneous mixture of the solvent and the clotting agent is formed.
